(19) United States
(12) Patent Application Publication
(10) Pub. No.: US 2014/0245658 A1
(43) Pub. Date: Sep. 4, 2014

(54) SYNTHESIS OF ETHANOL AND HIGHER ALCOHOLS BY HYDROGENATION OF KETENE

(71) Applicant: Pioneer Energy Inc, Lakewood, CO (US)

(72) Inventors: John T. Henri, Longmont, CO (US); Jan Zygmunt, Longmont, CO (US); Mark Berggren, Golden, CO (US); Robert Zubrin, Golden, CO (US)

(73) Assignee: PIONEER ENERGY INC, Lakewood, CO (US)

(21) Appl. No.: 14/175,880

(22) Filed: Feb. 7, 2014

Related U.S. Application Data
(60) Provisional application No. 61/762,405, filed on Feb. 8, 2013.

(51) Int. Cl. C07C 29/17; C07C 45/68; C07C 45/62; C07C 29/36; C10L 1/19; C07C 67/46

(52) U.S. Cl. CPC C07C 29/175; C10L 1/19; C07C 67/46; C07C 45/62; C07C 29/36; C07C 45/68
USPC 44/388; 585/310; 568/885; 560/265; 568/484; 568/408

(57) ABSTRACT

Ketene chemistry and hydrogenation reactions are used to synthesize fuels and chemicals. Ketene from acetic acid is hydrogenated to form fuels and chemicals; acetic acid can be synthesized from synthesis gas which is produced from coal, biomass, natural gas, etc. In one embodiment, the present application discloses methods to selectively synthesize higher alcohols and hydrocarbons useful as fuels and industrial chemicals from syngas and biomass.

### RELATED APPLICATIONS

This application claims the benefit of U.S. Provisional Application No. 61/762,405 filed Feb. 8, 2013 entitled “Synthesis of Ethanol and Higher Alcohols by Hydrogenation of Ketene” which is incorporated herein by reference.

### BACKGROUND OF THE INVENTION

Petroleum is a vital source of fuels for transportation, industrial chemicals that produce polymers, plastics, pharmaceuticals, paints and other important chemicals. However, due to economic, environmental and political factors, new sources besides petroleum are being sought for the production of these materials.

Technologies to manufacture industrial commodities such as methanol are mature and used to produce the world's supply from synthesis gas that comes from methane, coal, etc. Ethanol is produced by fermentation and hydration of ethylene produced from petroleum. Technology to produce ethanol from cellulose is still in development. Efforts have been made to develop a synthesis of ethanol from synthesis gas but no satisfactory technologies have materialized yet. There has been increased use of ethanol as a fuel additive and also as an automotive fuel itself.

Various efforts are underway to find economic fermentation processes to produce butanol which is also an important fuel alternative and industrial chemical. However the economy of these processes due to the time required for each fermentation cycle, environmental problems from large amounts of water consumption for these processes and difficulty of isolation of butanol from broths along with its toxicity to microbes are challenges that have not been overcome.

The present invention avoids these issues and is a novel method to synthesize fuels and chemicals from non-petroleum sources such as biomass, coal and natural gas. The synthetic manufacture of fuels and chemicals, for example alcohols and alkanes offers the potential for higher energy fuels that are readily compatible with existing automotive and transportation infrastructure.

### BRIEF DESCRIPTION OF DRAWINGS

FIG. 1 shows a representative method for the synthesis of ethanol by hydrogenation of ketene derived from acetone.

FIG. 2 shows a representative method for the synthesis of higher alcohols and hydrocarbons from acetic acid via ketene synthesized from acetic acid using a copper zinc oxide catalyst.

FIG. 3 shows a representative method for the synthesis of higher alcohols and hydrocarbons from acetic acid via ketene using a copper chromite catalyst.

FIG. 4 described in experiment 1 outlines an apparatus to form ketene from acetone using a ketene lamp, condense it and then pass it over a hydrogenation catalyst to form products.

FIG. 5 described in experiment 2 outlines an apparatus to form ketene and pass it through a hydrogenation reactor using vacuum applied at the outlet to form products.

FIG. 6 described in experiment 3 outlines an apparatus to form ketene and the use of argon sweep or inert gas to pass it through a hydrogenation reactor.

FIG. 7 described in experiment 4 outlines an apparatus to form ketene and pass it over hot glass beads in a heated stainless steel sample bottle and hydrogenate it in a reactor below.

FIG. 8 described in experiment 6 outlines an apparatus to form ketene from acetic acid and hydrogenate it.

FIG. 9 is a GCMS analysis of liquid product from experiment 1 showing ethanol and other products.

### DETAILED DESCRIPTION OF THE INVENTION

An important aspect of the present invention is a process to convert ketene to ethanol and other higher alcohols. Acetic acid is converted to ketene which may be hydrogenated to form ethanol. In another aspect, ketene dimerizes to diketene or undergoes further self addition reactions and is then reduced to form 1-butanol, 2-butanol, 2-heptanol, 4-heptanol and other higher alcohols, hydrocarbons or ketones.

The products of the present invention may be used as fuels. The products of the present invention may be used individually or as mixtures. The products of the invention may be used as fuels or blended with fuels such as gasoline, diesel, aviation or jet fuel or other fossil fuels. The products of the present invention may also be used as solvents and chemicals.

### Definitions

Unless specifically noted otherwise herein, the definitions of the terms used are standard definitions used in the art. Exemplary embodiments, aspects and variations are illustrated in the figures and drawings, and it is intended that the embodiments, aspects and variations, and the figures and drawings disclosed herein are to be considered illustrative and not limiting.

As used herein, an “alkyl” group is a straight, branched, cyclic, acylic, saturated or unsaturated, aliphatic group or alcoholic group having a chain of carbon atoms. A C₁-C₂₀ alkyl or C₁-C₂₀ alkanol, for example, may include alkyl groups that have a chain of between 1 and 20 carbon atoms, and include, for example, the groups methyl, ethyl, propyl, isopropyl, vinyl, allyl, 1-propenyl, isopropenyl, ethynyl, 1-propynyl, 2-propynyl, 1,3-butadienyl, penta-1,3-dienyl, penta-1,4-dienyl, hexa-1,3-dienyl, hexa-1,3,5-trienyl, and the like. An alkyl group may also be represented, for example, as a —(CR¹R²)ₘ— group where R¹ and R² are independently hydrogen or are independently absent, and for example, m is 1 to 8, and such representation is also intended to cover both saturated and unsaturated alkyl groups.

An “alkyl compound(s)” as used herein, is an alkyl containing 1 to 20 carbons (C₁-C₂₀ alkyl), and includes cyclic and acyclic alkanes, alkenes, alcohols, ketones and aromatics (e.g., benzene, toluene, ethyl benzene etc.) and mixtures thereof. The alkyl compound may be used as a raw material for chemical processing, a solvent or the alkyl compound may be used as a fuel or mixtures of fuels. Such fuel or mixtures of fuels may be further combined with other fuel or fuel products to form a gasoline. Non-exclusive examples of an alkyl compound include butane, 1-butanol, 2-butanol, 2-pentanol, 1-hexanol, 2-hexanol, 2-heptanol, 4-heptanol, 4-heptanone, 3-methyl cyclohexanol, 2,6-dimethyl-4-heptanol and mixtures thereof. Alkyl compounds of the present application exclude saturated beta-lactones.

A “cyclyl” such as a monocyclyl or polycyclyl group includes monocyclic, or linearly fused, angularly fused or bridged polycycloalkyl, or combinations thereof. Such cyclyl group is intended to include the heterocyclyl analogs. A cyclyl group may be saturated, partially saturated or aromatic.

An alkanol or an alcohol is a compound with an alkyl or cyclic alkyl group bearing a hydroxyl group. Examples of alcohols are methanol, ethanol, propanol, isopropanol, butanol (including 1-butanol, 2-butanol, isobutanol, tert-butanol), pentanol (and its isomers including 1-pentanol, 2-pentanol, 3-pentanol, isopentanol, neopentanol, cyclopentanol, etc) and straight chain, branched and cyclic isomers of other higher alcohols such as hexanol, cyclohexanol, methylcyclohexanol, heptanol (including 1-heptanol, 2-heptanol, 3-heptanol, 4-heptanol, iso-heptanol and other isomers), nonanol, etc. A higher alcohol is an alcohol having two or more carbons.

“Gasoline” is known to comprise of a complex mixture of volatile hydrocarbons suitable for use as a fuel in a spark-ignition internal combustion engine. Typically, gasoline boils over a range of about 27° C. to about 225° C. Gasoline may consist of a single blendstock, such as the product from a refinery alkylation unit, or it may comprise of a blend of several blendstocks. The blending of gasoline is well known in the art and may include a combination of three to twelve or more different blendstocks. Optimization of the blending process takes into account a plurality of characteristics of both the blendstocks and the resulting gasoline, and may include such characteristics as cost and various measurements of volatility, octane, boiling point characteristics and chemical composition. While hydrocarbons usually represent a major component of gasoline, certain oxygen containing organic compounds may be included as gasoline components. In one aspect, such oxygen containing organic compounds are referred to as “oxygenate” or “oxygenates,” and are important gasoline substitutes such as ethanol and butanol. Oxygenates are also useful as components in gasoline because they are usually of high octane and can be a more economical source of gasoline octane than a high octane hydrocarbon blending component such as alkylate or reformate. Oxygenates that may be used as gasoline blending agents include, but are not limited to, methanol, ethanol, tertiary-butyl alcohol, methyl tertiary-butyl ether, ethyl tertiary-butyl ether and methyl tertiary-amyl ether. Various catalysts may be used in reduction or hydrogenation reactions of the present application. The catalyst used may contain one or more transition metal such as ruthenium, palladium, platinum, rhodium, nickel, iridium, rhenium, copper, zinc, chromium, nickel, iron, cobalt or combinations of thereof. The catalyst may contain a combination of one or more transition metals with main group elements such as for example platinum and tin or ruthenium and tin. The catalyst may contain promoters such as oxides of barium, magnesium, etc. Reduction or hydrogenation may also be done using Raney type sponge catalysts such as Raney nickel, copper, cobalt, etc optionally bearing promoters such as iron, molybdenum, chromium, palladium, etc.

Catalysts used in reductions may be supported or unsupported. A supported catalyst is one in which the active metal or metals are deposited on a support material; e.g. prepared by soaking or wetting the support material with a metal solution, spraying or physical mixing followed by drying, calcination and finally reduction with hydrogen if necessary to produce the active catalyst. Catalyst support materials used frequently are porous solids with high surface areas such as silica, alumina, titania, magnesia, carbon, zirconia, zeolites etc.

The methods of the present invention can comprise, consist of, or consist essentially of the essential elements and limitations of the method described herein, as well as any additional or optional ingredients, components, or limitations described herein or otherwise useful in synthetic organic chemistry.

“Synthesis gas” or “syngas” is a mixture of varying amounts of carbon monoxide and hydrogen. Syngas maybe produced by the partial oxidation of materials such as methane, liquid hydrocarbons, coal, biomass, etc.

“Biomass” is material obtained from living or recently living organisms.

Acetic acid may be made by oxidation of ethanol produced by fermentation or conversion of synthesis gas to methanol followed by its carbonylation. Synthesis gas may be obtained in large quantities from biomass, coal, natural gas, etc.

In one embodiment acetic acid used in the invention is made from syngas. Syngas may be made from coal, natural gas or like fossil fuel. In another embodiment the acetic acid used in the invention is synthesized from syngas made from a renewable source such as biomass from corncobs, switchgrass, wood chips, recyclable materials like agricultural waste, land fill materials, industrial waste, municipal solid waste, sewage and the like.

Ketene may be synthesized by various methods described in literature such as dehydration of acetic acid, pyrolysis of acetone or acetic anhydride, etc. In one embodiment ketene is synthesized from acetone. In another embodiment ketene is synthesized from acetic acid. In one embodiment, the ketene produced is dimerized to produce diketene which can be used directly in reduction steps or stored before reduction.

In other embodiments, there are provided methods for preparing alcohols from ketene. In one embodiment there is a method to synthesize ethanol comprising the steps of synthesizing ketene and hydrogenating ketene in the presence of a catalyst to produce ethanol.

In one embodiment the catalyst used in hydrogenating ketene is a transition metal based catalyst. In one embodiment the catalyst employed in hydrogenation comprises of two or more transition metals. In one embodiment the catalyst employed in hydrogenation comprises a copper zinc oxide based catalyst. In one embodiment, the catalyst employed in hydrogenation comprises a copper chromite based catalyst.

In another embodiment, a method is described to synthesize a mixture of higher alcohols by synthesizing ketene, reacting ketene in self addition reactions and then passing it over a hydrogenation catalyst with hydrogen to produce said higher alcohols.

In one embodiment ketene is passed over a hot surface before hydrogenation to promote higher alcohol synthesis.

In one embodiment, ketene is subjected to compression before hydrogenation to promote formation of higher alcohol products. In one embodiment, ketene is subjected to compression and heating before hydrogenation to promote formation of higher alcohol products.

In one embodiment, ketene is subjected to compression before hydrogenation to promote formation of higher alcohol products. In one embodiment, ketene is subjected to compression before hydrogenation to promote formation of higher alcohol products.

In one embodiment, ketene is passed through a container with volume to increase its residence time and promote quick dimer formation and then hydrogenated to form higher alcohols.

In one embodiment, an ester is a product of the hydrogenation of ketene. In one embodiment, the ester is further hydrogenated to form a product selected from the group of alcohol, ketone, aldehyde or hydrocarbon product.

In one embodiment, a ketone is a product of the hydrogenation of ketene. In one embodiment, the ketone is further hydrogenated to form a alcohol or hydrocarbon product. In one embodiment, the ketone is 2-butanone. In one embodiment, the ketone is 2-heptanone or 4-heptanone.

In one embodiment, an aldehyde is a product of the hydrogenation of ketene. In one embodiment, the aldehyde is further hydrogenated to form a alcohol or hydrocarbon product. In one embodiment, the aldehyde is butyrldehyde.

In one embodiment of the process, the hydrogenation catalyst comprises of one or more transition metals. In one embodiment, the catalyst is a copper zinc based catalyst. In one embodiment, the catalyst is a copper chromite based catalyst. In one embodiment, the catalyst comprises of a mixture of transition metal catalysts and main group elements.

In one embodiment, there is provided a method to synthesize a fuel by synthesizing ketene and hydrogenating the ketene in the presence of a catalyst to produce said fuel. In one embodiment, the fuel is blended with a hydrocarbon mixture derived from a petroleum source. In one embodiment, the synthesized fuel is blended with a biofuel. In one embodiment, the synthesized fuel is blended with ethanol derived from a fermentation process.

In one embodiment, there is provided a method to synthesize alcohols by the steps of a) synthesizing methanol from synthesis gas, b) carbonylating methanol to form acetic acid, c) synthesizing ketene from acetic acid and d) hydrogenating of said ketene in the presence of a catalyst to produce said alcohols.

In one embodiment, the alcohols produced are dehydrated to form unsaturated hydrocarbon products. In one embodiment, the unsaturated hydrocarbons are dimerized, trimerized or oligomerized to form higher molecular weight products. In another embodiment, unsaturated hydrocarbons are hydrogenated to form saturated hydrocarbons.

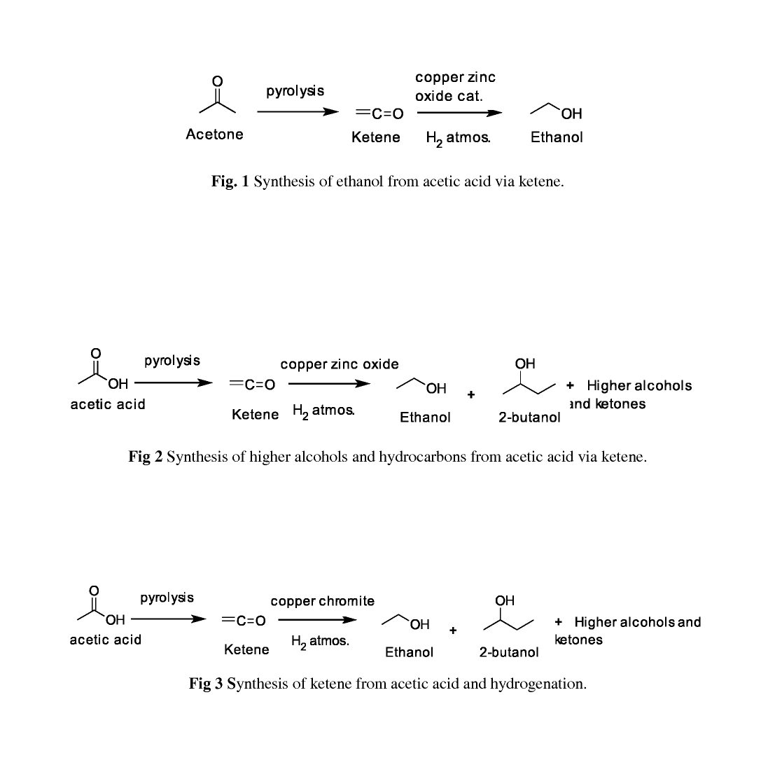

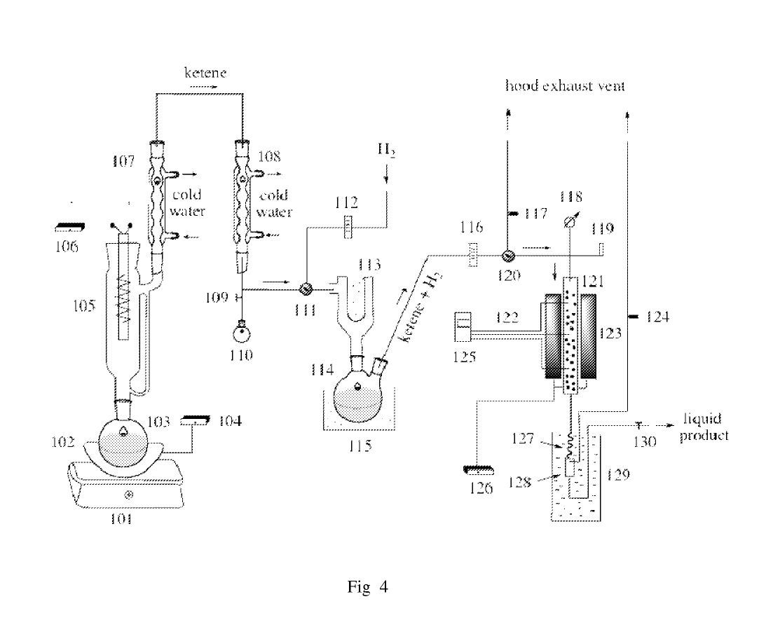

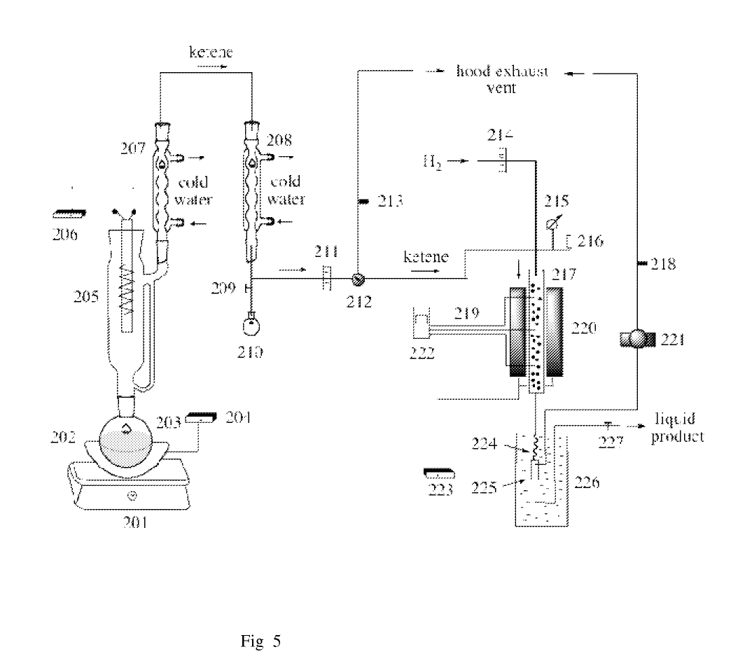

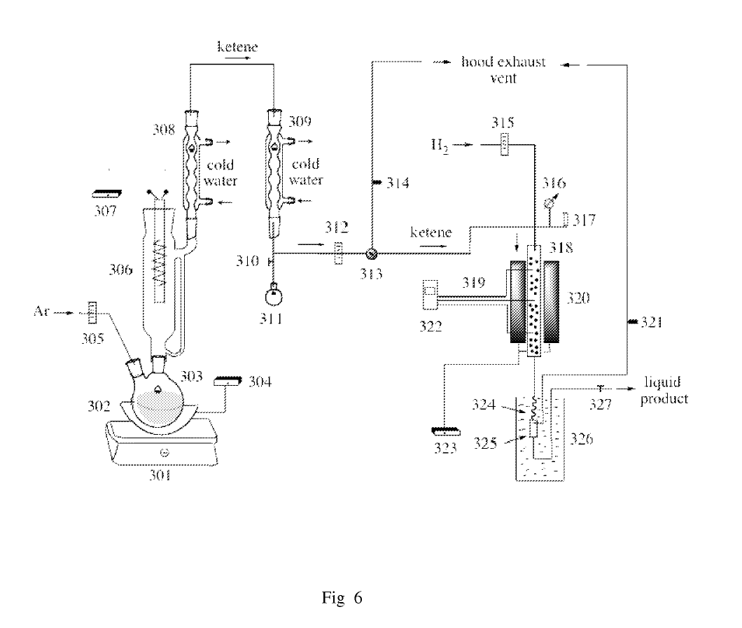

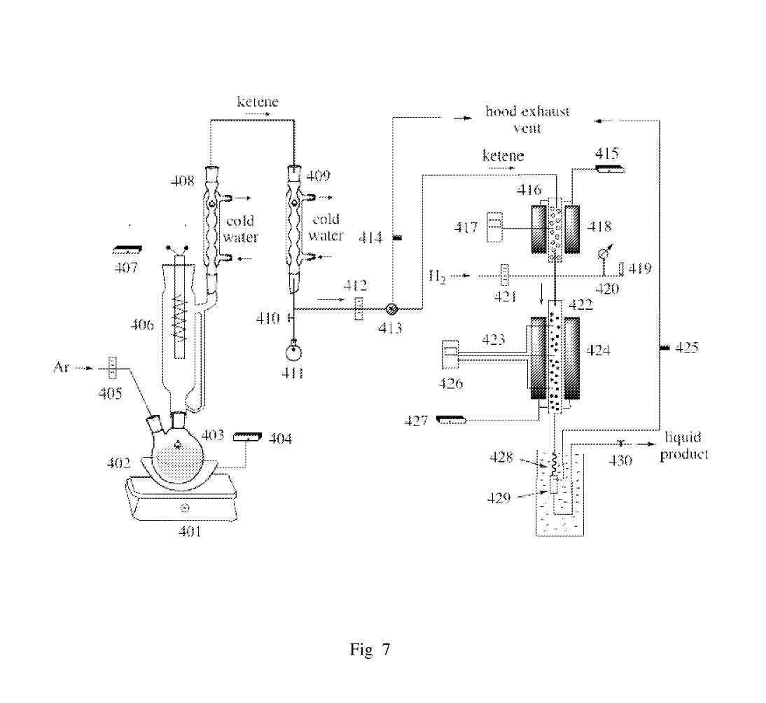

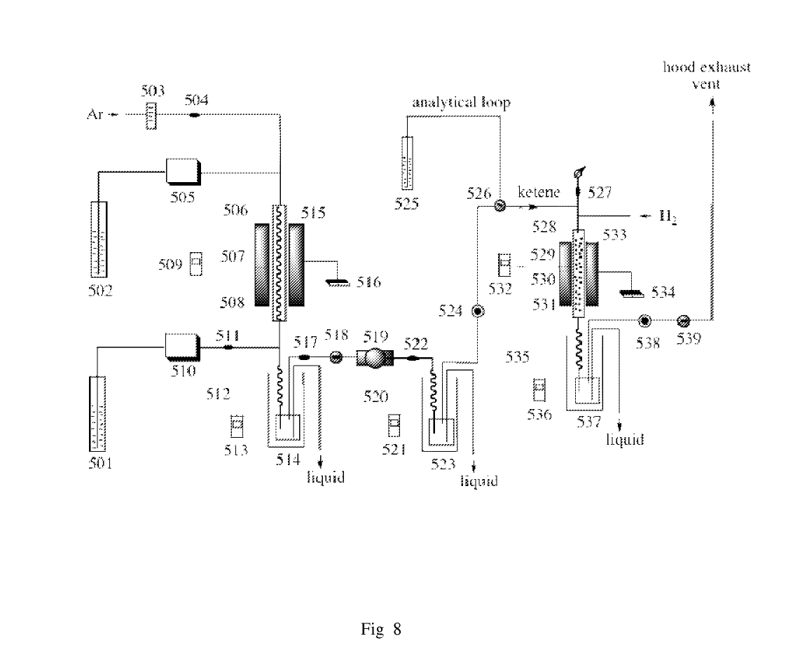

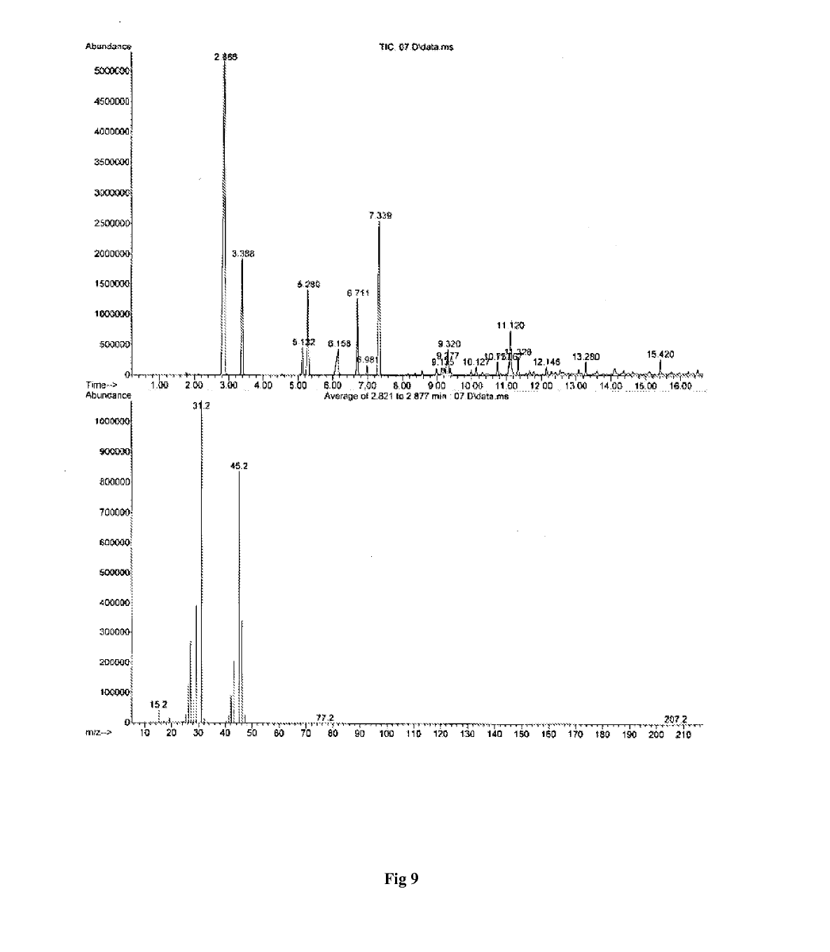

In one variation, the compounds may be synthesized by the steps outlined in FIG. 1, FIG. 2, FIG. 3, FIG. 4, FIG. 5, FIG. 6, FIG. 7, FIG. 8, FIG. 9 or FIG. 10.

### EXPERIMENTAL

The following procedures may be employed for the preparation of the compounds of the present application. The starting materials and reagents used in preparing these compounds are either available from commercial suppliers such as SigmaAldrich, Alfa Aesar, etc or are prepared by methods well known to a person of ordinary skill in the art, following procedures described in such references as Fieser and Fieser's Reagents for Organic Synthesis, vols. 1-17, John Wiley and Sons, New York, N.Y., 1991; Rodd's Chemistry of Carbon Compounds, vols. 1-5 and supps., Elsevier Science Publishers, 1989; Organic Reactions, vols. 1-40, John Wiley and Sons, New York, N.Y., 1991; March J.: Advanced Organic Chemistry, 4th ed., John Wiley and Sons, New York, N.Y.; and Larock: Comprehensive Organic Transformations, VCH Publishers, New York, 1989. Standard organic chemical reactions can be achieved by using a number of different reagents, for example, as described in Larock: Comprehensive Organic Transformations, VCH Publishers, New York, 1989.

Samples were analyzed on a Agilent 6890 5973 GCMS system equipped with a JW1 DB624 column with dimensions of 30 m×250μ×1.4μ. The method ran at 1 ml/min flow, with oven temperature at 40° C. for the first two minutes followed by temperature ramp at 10° C./min to a temperature of 240° C. which was held for 10 minutes. The solvent delay was set at 5 minutes. Chemical identities of compounds were confirmed by mass spectroscopic qualitative analysis on GCMS against a NIST 2011 library as well as by comparison of retention time against commercial standards. Analysis of samples with volatile alcohols was done on a Gowmac GC system using a Hayesep Q column with He and nitrogen carrier gases. Column temperature was 180° C., detector temperature was 150° C., injector temperature was 150° C., sample valve temperature was 105° C. detector current was 107 mA and injection volume was 1 microliter. Gas samples were analyzed on Varian Micro-GC instruments.

The quality of GCMS NIST library matches are listed on the last column of tables. Apparatus used for hydrogenation reactions was fabricated in house using standard pipes and parts and instruments available from companies such as Swagelok, Omega engineering, etc. Glassware was purchased commercially or fabricated.

Ketene was generated by pyrolysis of acetone or acetic acid.

### Experiment 1

Synthesis of Ketene and Hydrogenation: A 1 lit RB flask 103 was filled with about 600 ml of acetone, placed on a heating mantle 102 on a magnetic stirrer 101 and a ketene lamp 105 was fitted on the neck of the flask (see FIG. 4). The top of the ketene lamp was connected to two reflux condensers 107 and 108 connected in series with the bottom of the second connected to a 50 ml RB flask 110 with a side arm from the bottom for ketene gas to flow to an outlet with a hose connector to a Dewar condenser 113,114,115. The connection between the ketene lamp and the Dewar condenser was fitted with a three way valve 111. The valve allowed switching of flow from the ketene reactor to flow of hydrogen gas from a hydrogen cylinder. The hydrogen line was fitted with a rotometer 112. The outlet from the Dewar condenser could be vented to the hood outlet or into a hydrogenation reactor. The hydrogenation reactor consisted of a 1 inch stainless steel tube 121 fitted with thermocouples 122 inside and outside wrapped with a heating rope 123 which was covered with insulation. The outlet from the ketene reactor which carried generated ketene was fitted with a rotometer 116 and connected to the top inlet of the hydrogenation reactor. A pressure gauge 118, apres sure safety valve 189 were connected to the top of the hydrogenation reactor. The bottom of the hydrogenation reactor tube was connected to a ⅛′ coil of stainless steel tubing 127 about 50 inches long the other end of which was connected to a 2 inch length of ½ diameter tube 128 to collect liquid condensate. The bottom of the tube had a liquid draining tube with a ball valve at its end 130. The top of the ½ inch tube had a ⅛ inch tube including GC port 124 which vented gas from the system to the hood vent. Two GC ports 117 and 124 were fixed after the ketene reactor and after the hydrogenation reactor to analyze gas samples of the ketene feed and hydrogenation product.

Dry ice was added to the Dewar condenser and cold water was circulated through the ketene lamp reflux condensers and the lamp was started. After refluxing acetone from 5 minutes, the heating filament of the ketene lamp was turned on till red hot to start ketene generation. Ketene was condensed in the Dewar for about 1 hour and the lamp shut off. The 3 way valve was then turned to cut ketene gas flow and start hydrogen gas sweep at ˜200 ml/min over the condensed ketene in the flask 115 under Dewar condenser into the hydrogenation reactor. The hydrogenation reactor contained 77.5 gms of Copper Zinc oxide catalyst from Unicat (LS-402) which had been reduced overnight with 5% hydrogen/95% argon at 195° C. overnight followed by 1 hour reduction with hydrogen before ketene gas was introduced for reduction. The coiled condenser at the bottom of the reactor was cooled with an ice salt water bath. Gas samples from a GC port before the vent to atmosphere were collected from the ketene reactor outlet as ketene generation was in progress indicating by GCMS that major ketene and methane along with lesser amounts of CO and ethylene was generated. Gas samples were also collected at the gas outlet of the hydrogenation reactor through a GC port. GCMS of samples indicated complete reduction of ketene and product peaks containing ethanol, ethylacetate, 2-butanone, 2-butanol, 1-butanol and pentanol among other products. After ketene/hydrogen gas was passed through the hydrogen reactor for about an hour a liquid sample was collected weighing 0.35 gm of liquid. GC of the sample indicated 55.6% ethanol, 6.1% 2-butanol, 5.6% ethyl acetate by volume against standards analyzed on the Gowmac GC.

Other products seen on GCMS (qualitive analysis) are shown in table 1.

TABLE 1

| | | Peak area |
|---|---|---|
| ~RT, min | Compound | 60 min sample |
| 2.89 | ethanol | 51.1 |
| 5.13 | ethyl acetate | 2.3 |
| 5.28 | 2-butanol | 7.5 |
| 6.16 | acetic acid | 4.8 |
| 6.71 | 1-butanol | 7.2 |
| 6.98 | 2-pentanone | 0.8 |
| 7.34 | 2-pentanol | 15.8 |
| 9.28 | 3-hexanol | 0.9 |
| 9.32 | acetic acid butyl ester | 1.9 |
| 10.74 | 4-heptanone | 1.0 |
| 11.12 | 4-heptanol | 2.8 |
| 11.33 | 2-heptanol | 1.5 |
| 13.28 | 3-ethyl-4-heptatnone | 1.1 |
| 15.42 | 1,3-butanediol diacetate | 1.2 |

### Experiment 2

Synthesis and Hydrogenation of Ketene: A 1 lit RB flask 203 was filled with about 600 ml of acetone, placed on a heating mantle 202 on a magnetic stirrer 201 and a ketene lamp 205 was fitted on the neck of the flask (see FIG. 5). The top of the ketene lamp was connected to two reflux condensers 207 and 208 connected in series with the bottom of the second connected to a 50 ml RB flask 210 with a side arm from the bottom of the condenser for ketene gas to flow to an outlet with a hose connector to a silicone tube connected to a three way ball valve 212. One out from the ball valve was connected to a hose that vented to the hood exhaust and the other out from the ball valve 212 was connected to a hydrogenation reactor inlet. A rotometer 211 was connected to measure ketene gas/methane from the ketene lamp as it flowed into the hydrogenation reactor or vented to the exhaust. The hydrogenation reactor which was connected at its top to a hydrogen gas line with a rotometer 214 to measure hydrogen flow in. The other parts of the hydrogenation reactor were as described in the experiment above i.e. it was a steel tube 217 equipped with a liquid condenser 226 at its bottom, pressure gauge 215, pressure relief valve 216 with thermocouples 219 to measure temperature and heating rope and insulation 220 to maintain elevated temperatures for the reaction. Two GC ports 213 and 218 were installed after the ketene reactor and after the hydrogenation reactor to sample ketene gas feed and hydrogenation product. The hydrogenation reactor contained reduced copper zinc oxide catalyst used in the experiment above. The acetone was refluxed and ketene filament heated to begin ketene gas generation. Ketene was vented to the exhaust for about 10 minutes to ensure a steady rate and GCMS samples were taken of the gas indicating ketene was formed along with byproduct methane and some uncondensed acetone vapor. Hydrogen gas was passed through the heated hydrogenation reactor at 200 ml/min at ˜205° C. A manual vacuum pump 221 was attached to the hydrogenation reactor gas outlet and the three way ball valve was turned to admit ketene gas into the hydrogenation reactor as a very low pressure was applied. Ketene flowed in a undulating fashion at a rate of 0 to 300 ml/min. GCMS samples indicated all ketene was reduced and ethanol and 2-butanol were the major products along with other higher alcohols, ketones and ethyl acetate. Acetone and isopropanol were observed in the samples resulting from acetone evaporation and feed from the acetone in the ketene lamp. After about 1 hour, 3.5 gms of liquid product was collected from the liquid condenser cooled in an ice salt water bath. Quantative GC analysis of the sample showed about 22% ethanol and 7.7% 2-butanol as major products along with acetone/isopropanol a major portion of the rest of the liquid volume. The reaction was stopped overnight and restarted again for 1 hour and then utilizing very low vac pump instead of a hand vacuum pump for another hour. After the first 1 hour, 2.25 gms of liquid sample was collected containing 24% ethanol and 9% 2-butanol by volume. A sample after the second hour contained 29.6% ethanol and 8.8% 2-butanol by volume. Major constituents of the remaining volume in the two samples were acetone and isopropanol which was present in the ketene gas feed as a result of uncondensed acetone from the ketene lamp. GCMS of sample 2 is shown in table 2.

TABLE 2

| | | Peak area |
|---|---|---|
| RT, min | Compound | 120 min sample |
| 2.87 | ethanol | 45.76 |
| 5.07 | 2-butanone | 2.07 |
| 5.14 | ethyl acetate | 23.60 |
| 5.27 | 2-butanol | 2.90 |
| 6.11 | i-propyl acetate | 4.76 |
| 6.69 | 1-butanol | 1.66 |
| 6.98 | 2-pentanone | 7.45 |
| 7.31 | 2-pentanol | 7.45 |
| 9.00 | butyric acid ethyl ester | 1.24 |
| 9.32 | acetic acid butyl ester | 1.66 |
| 10.74 | 4-heptanone | 1.45 |

### Experiment 3

Synthesis and Hydrogenation of Ketene: The ketene generation apparatus was as described in the experiment above except that the 1 liter, one neck RB flask was replaced by a two neck flask 303 (see FIG. 6). The side neck was connected to a ground glass outlet that was connected to a rotometer 305 on a argon line to sweep the argon gas into the hydrogenation reactor instead of applying vacuum. The ketene lamp was started after the acetone was refluxing and ketene vented to the exhaust to establish a steady rate for about 10 minutes. GCMS samples were taken indicating ketene, methane and some acetone vapor was in the product from the ketene lamp. The hydrogenation reactor containing 77 gms of reduced Copper zinc oxide catalyst was heated to about 200° C. and hydrogen flow was set to 300 ml/min as the ketene gas was passed through the reactor for hydrogenation. The liquid condenser was cooled in an ice salt water bath and gas and liquid samples were taken. The samples showed complete reduction of ketene to form ethanol and 2-butanol. GCMS analysis of samples after 30 and 60 minutes are shown in table 3.

TABLE 3

Peak area

| | | 30 min | 60 min |
|---|---|---|---|
| ~RT, min | Compound | sample | sample |
| 2.84 | ethanol | 14.4 | 21.5 |
| 5.07 | 2-butanone | 2.6 | 5.1 |
| 5.14 | ethyl acetate | 25.0 | 37.5 |
| 5.27 | 2-butanol | 3.2 | 2.7 |
| 6.10 | i-propyl acetate | 6.9 | 8.0 |
| 6.98 | 2-pentanone | 12.1 | 13.6 |
| 7.31 | 2-pentanol | 11.0 | 5.8 |
| 8.09 | methyl i-butyl ketone | 5.8 | 1.5 |
| 8.57 | 4-methyl-2-pentanol | 3.7 | 0.0 |
| 9.06 | 2-methyl-4-heptanol or isomer | 0.0 | 0.0 |
| 9.32 | acetic acid butyl ester | 1.5 | 2.2 |
| 10.74 | 4-heptanone | 3.2 | 2.2 |
| 10.94 | 4-methyl-2-pentanol acetate | 0.0 | 0.0 |
| 11.67 | 2-methyl-4-heptanone | 6.3 | 0.0 |
| 12.55 | 2,6-dimetyl-4-heptanone | 4.3 | 0.0 |

### Experiment 4

Synthesis and Hydrogenation of Ketene: A 1 lit 2 necked flask containing ˜300 ml of acetone attached to a ketene lamp by the main neck and to an argon line with a rotometer 405 connected to the side neck (see FIG. 7). The ketene lamp was started after acetone was refluxing and the ketene swept from the lamp into the hydrogenation reactor with a sweep of ˜30 ml/min argon. A stainless steel sample bottle 416 (Hoke bottle) containing 150 gms of 5 mm borosilicate glass beads heated by heating rope 418 to about 150° C. was connected to the hydrogenation reactor 422 heated by heating rope 424. The hydrogenation reactor contained 77 gms of pre-reduced copper-zinc oxide catalyst (LS-402, Unicat) as described in the previous experiments. The ketene gas was passed over the hot glass beads and hydrogen gas via a line with a rotometer 421 introduced at ˜300 ml/min into the reactor after the Hoke bottle on the top of the reactor as described in the FIG. 7 below. Gas samples were collected from GC ports 414 and 425 after the ketene reactor and after the hydrogenation reactor and liquid samples from the condenser 428/429 including valve 430. The hydrogenation reactor was at 187° C. and rose to 246° C. as the reaction progressed. The compounds in the GCMS samples are described in table 4.

TABLE 4

| | | Peak area | | |
|---|---|---|---|---|
| | | 40 min | 60 min | 80 min |
| ~RT, min | Compound | sample | sample | sample |
| 2.81 | ethanol | 4.0 | 1.8 | 2.6 |
| 5.07 | 2-butanone | 1.7 | 2.8 | 3.9 |
| 5.13 | ethyl acetate | 9.9 | 19.8 | 36.5 |
| 5.26 | 2-butanol | 2.8 | 1.3 | 0.0 |
| 6.10 | i-propyl acetate | 14.2 | 40.5 | 26.1 |
| 6.97 | 2-pentanone | 1.7 | 3.1 | 5.2 |
| 7.30 | 2-pentanol | 1.9 | 0.0 | 0.0 |
| 8.09 | methyl i-butyl ketone | 5.9 | 4.6 | 3.5 |
| 8.16 | sec-butanol acetate | 0.0 | 1.0 | 0.0 |
| 8.22 | acetic anhydride | 0.0 | 1.8 | 13.9 |
| 8.57 | 4-methyl-2-pentanol | 7.6 | 1.8 | 0.0 |
| 9.06 | 2-methyl-4-heptanol or | 7.3 | 0.0 | 0.0 |
| | isomer | | | |
| 10.94 | 4-methyl-2-pentanol | 0.0 | 2.6 | 0.0 |
| | acetate | | | |
| 11.67 | 2-methyl-4-heptanone | 1.4 | 1.8 | 0.0 |
| 12.55 | 2,6-dimetyl-4-heptanone | 24.1 | 13.9 | 8.4 |
| 13.08 | 2,6-dimethyl-4-heptanol | 11.3 | 3.1 | 0.0 |
| 13.68 | 2,6-dimethyl decane | 6.1 | 0.0 | 0.0 |

### Experiment 5

Synthesis and Hydrogenation of Ketene over a Copper Chromite Type Catalyst: A 1 liter RB flask filled with ˜400 ml of acetone placed on a heating mantle/stir plate was connected to a ketene lamp as described in experiment 1 above and FIG. 4 below. The hydrogenation reactor was filled with 81 gms of copper chromite catalyst (Strem Chemicals 29-0410) which was reduced at ˜195 ° C. with 5% hydrogen/95% argon overnight and hydrogen for one hour. Acetone reflux was started and after five minutes the ketene lamp was started and ketene condensed after the Dewar condenser and flask below were cooled to −78 in acetone dry ice.

GCMS samples of ketene product indicated a steady generation of ketene which was condensed by the Dewar for about 2.5 hours. The ketene lamp was switched off and hydrogen gas was swept over the condensed ketene at about 400 ml/min. Ketene reduction was started around 205° C. An exotherm was observed as temperature rose to 212° C. GCMS analysis indicated mostly gaseous products like methane, CO₂, ethane and propane. The temperature of the reactor was decreased as samples were taken and analysis done by GCMS. As the temperature went below 90° C., ethanol and ethyl acetate was observed in product GC analysis.
See table 5 for GCMS analysis of gas samples taken at 212, 205, 174, 136, 90, 73 and 60° C.

TABLE 5

| | | Peak area of samples as temperature decreased | | | | | | |
|---|---|---|---|---|---|---|---|---|
| ~RT, | | 212° | 205° | 174° | 136° | 90° | 73° | 60° |
| min | Compound | C. | C. | C. | C. | C. | C. | C. |
| 1.34 | CH₄ + CO | 3.1 | 19.1 | 4.6 | 9.0 | 7.3 | 1.3 | 0.0 |
| 1.38 | CO₂ | 37.2 | 19.0 | 25.9 | 40.3 | 7.3 | 6.2 | 0.0 |
| 1.40 | ethane | 19.6 | 41.9 | 19.8 | 0.0 | 0.0 | 0.0 | 0.0 |
| 1.50 | ketene | 0.0 | 0.0 | 0.0 | 0.0 | 0.0 | 0.0 | 87.4 |
| 1.52 | propane | 16.2 | 8.8 | 24.0 | 28.1 | 10.2 | 8.3 | 0.0 |
| 1.64 | allene | 0.0 | 0.0 | 0.0 | 0.0 | 0.0 | 0.0 | 2.3 |
| 1.71 | i-butane | 3.0 | 3.1 | 5.9 | 6.9 | 0.0 | 0.0 | 0.0 |
| 1.87 | n-butane | 12.4 | 8.0 | 11.1 | 2.4 | 9.3 | 16.5 | 7.0 |
| 1.91 | 1,2- | 0.0 | 0.0 | 0.0 | 0.0 | 0.0 | 0.0 | 3.3 |
| | butadiene | | | | | | | |
| 2.42 | 2-Me- | 1.0 | 0.0 | 1.3 | 1.4 | 0.0 | 0.0 | 0.0 |
| | butane | | | | | | | |
| 2.70 | pentane | 5.1 | 0.0 | 2.6 | 1.0 | 0.0 | 0.0 | 0.0 |
| 2.80 | ethanol | 0.0 | 0.0 | 0.0 | 1.8 | 27.9 | 14.4 | 0.0 |
| 3.70 | 2-Me- | 1.5 | 0.0 | 2.6 | 1.1 | 0.0 | 0.0 | 0.0 |
| | pentane | | | | | | | |
| 4.26 | n-hexane | 0.9 | 0.0 | 2.2 | 1.4 | 0.0 | 0.0 | 0.0 |
| 5.13 | ethyl acetate | 0.0 | 0.0 | 0.0 | 1.5 | 35.2 | 48.0 | 0.0 |
| 6.10 | i-propyl | 0.0 | 0.0 | 0.0 | 0.0 | 0.0 | 5.3 | 0.0 |
| | acetate | | | | | | | |
| 8.21 | acetic | 0.0 | 0.0 | 0.0 | 5.0 | 2.7 | 0.0 | 0.0 |
| | anhydride | | | | | | | |

### Experiment 6

Synthesis of Ketene from Acetic Acid and Hydrogenation. A reactor (FIG. 8) to synthesize ketene was set up in a fume hood. Acetic acid contained in measuring cylinder 502 was fed with HPLC pump 505 into ketene reactor consisting of coiled ⅜′ stainless steel tube 6′ long placed into a cylindrical heating furnace 515. Argon gas was fed along with the acetic acid to the top of the reactor and controlled by a ball valve and metered via rotometer 503. Thermocouples 506, 507, 508 were placed inside at the top and bottom of the reactor to monitor reaction temperature and also in the furnace cavity to control heating with controller 516. A 1/16″ tube conveying an ammonia solution from measuring cylinder 501 was introduced just after the ketene reactor outlet. The tube was then connected to liquid condenser 514 bearing a 6′ long ⅜″ steel tube coil which emptied into a 250 ml steel cylinder with a gas outlet at its top to convey ketene out and a liquid outlet tube exiting the bottom of the cylinder. The condenser 514 was immersed in an ice salt water bath. The condenser outlet was connected to a needle valve 518 which was connected to a vacuum pump 519. Pressure transducers were placed before the entry of the ketene reactor 504, at the outlet of the reactor 511, after condenser 1, 517 and after vacuum pump 522 to monitor vacuum and pressure of the process. The outlet of the vacuum pump was connected to a second liquid condenser 523 similar to condenser 514 described above. The outlet of condenser 524 was a tube bearing a GC port 524, pressure gauge and a pressure release valve followed by a three way valve one outlet of which was connected to a T. The other end of the three way valve was connected to a bubbler 525 containing an aniline solution in methanol used for quantitative analysis of ketene production at periodic short intervals during the process. One end of the T was connected to a 1″ steel tube reactor wrapped in heating tape and insulation bearing three thermocouples inside 528, 529, 531 and one on outer shell of reactor 530 to monitor its temperature. The other end of the T is connected to a hydrogen gas feed bearing a check valve and pressure gauge. The outlet of the hydrogenation reactor was connected to a condenser 537 similar to condenser 514. Gas outlet of condenser 537 was connected to a tube bearing a GC port 538 and back pressure valve and pressure gauge 539.

Argon flow was set at 1250 ml/minute and vacuum at 430 mbar for the system as the furnace heating was set to 700° C. for top and 750° C. for the bottom. Acetic acid containing 0.3% triethyl phosphate was pumped at a rate of 1.6 ml/minute through the HPLC pump into the ketene reactor that got to about 580° C. for reactor top and 770° C. for reactor bottom temperature. The HPLC pump to neutralize triethyl phosphate was turned on. The condenser baths were cooled with ice/salt water. GC samples and 100 ml aniline solutions were used to measure ketene production as the three way valve 526 turned to vent product until steady gas flow was achieved. The hydrogenation reactor containing reduced Cu—ZnO catalyst was heated to about 200° C. as hydrogen was passes at about 950 ml/min. Products from hydrogenation reactor were analyzed by GCMS shown in table. Due to evaporation of liquids in a high gas flow stream, a Dewar condenser was used to condense liquid.

TABLE 6

| | | % of total |
|---|---|---|
| RT, min | Compound | 90 min sample |
| 2.00 | Acetaldehyde | 14.76 |
| 2.75 | Pentane | 0.83 |
| 2.79 | Ethanol | 6.09 |
| 3.23 | Acetone | 14.24 |
| 3.77 | 2-methyl pentane | 0.87 |
| 4.92 | 1-butanal + methyl vinyl ketone | 4.37 |
| 5.10 | 2-butanone | 7.08 |
| 5.16 | acetic acid ethyl ester | 15.35 |
| 5.28 | 2-butanol | 0.17 |
| 5.93 | acetic acid | 1.75 |
| 6.13 | acetic acid i-propyl ester | 0.84 |
| 6.42 | Heptane | 1.29 |
| 7.03 | 2-pentanone | 7.97 |
| 8.25 | acetic anhydride | 7.87 |
| 8.32 | 3-penten-2-one | 1.59 |
| 9.40 | acetic acid butyl ester | 1.63 |
| 10.85 | 4-heptanone | 0.89 |
| 11.27 | 2-heptanone | 0.77 |

While a number of exemplary embodiments, aspects and variations have been provided herein, those of skill in the art will recognize certain modifications, permutations, additions and combinations and certain sub-combinations of the embodiments, aspects and variations. It is intended that the following claims are interpreted to include all such modifications, permutations, additions and combinations and certain sub-combinations of the embodiments, aspects and variations are within their scope. The entire disclosures of all documents cited throughout this application are incorporated herein by reference.

### REFERENCES

1. Organic Synthesis, Coll. Vol. 3, pp 508 (1955).
2. Organic Synthesis, Vol. 20, pp 26 (1940). Organic Synthesis, Coll. Vol. 3, pp 508 (1955).

### 3. The Chemistry of Ketenes, Allenes and Related Compounds, Part 1, 292 John Wiley and Sons, New York (1980).

4. Recent advances in process and catalysis for the production of acetic acid, Appl. Cat. A: General, 221, 253-265, 2001.

What is claimed is:

1. A method for the preparation of an alkyl compound or a mixture of alkyl compounds, the method comprising:
a) preparing a ketene of the formula I: 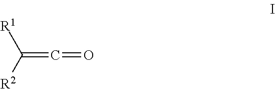
wherein:
R^{1 is H or a C}₁₋₆ alkyl group; and
R^{2 is H or a C}₁₋₆ alkyl group; and
b) hydrogenating the ketene of the formula I in the presence of a metal catalyst to form the alkyl compound or the mixtures of alkyl compounds.

2. The method of claim 1, wherein R¹ and R² are hydrogen.

3. The method of claim 1, wherein the metal catalyst is a transition metal.

4. The method of claim 1, wherein the metal catalyst is selected from the group consisting of a copper catalyst, a nickel catalyst and a zinc catalyst, or a mixture of copper, nickel and zinc catalyst.

5. The method of claim 1 wherein the product alkyl compound is ethanol.

6. The method of claim 1, wherein the product alkyl compound is a C₄ alcohol selected from 1-butanol and 2-butanol or mixtures thereof.

7. The method of claim 1, wherein the product alkyl compound is selected from the group consisting of ethanol, isopropanol, 1-butanol, 2-butanol, 2-pentanol, 1-hexanol, 2-hexanol, 2-heptanol, 4-heptanol, 3-methyl cyclohexanol, 2,6-dimethyl-4-heptanol and mixtures thereof.

8. The method of any one of claim 1, wherein the product alkyl compound or mixtures thereof is further purified to produce a fuel.

9. The method of claim 1, wherein the catalyst is selected from the group consisting of a copper chromite based catalyst, a copper oxide zinc oxide based catalyst, a nickel on alumina based catalyst and a ruthenium based catalyst, or mixtures thereof.

10. The method of claim 1 wherein the alkyl compound is an ester.

11. The method of claim 10 where said ester is further hydrogenated to form a product selected from the group of an alcohol, an aldehyde or a hydrocarbon.

12. The method of claim 1 wherein the alkyl compound is a ketone.

13. The method of claim 12 where said ketone is further hydrogenated to form an alcohol or a hydrocarbon.

14. The method of claim 12 where said ketone is selected from the group consisting of 2-butanone, 2-pentanone, 2-heptanone and 4-heptanone.

15. A method to synthesize ethanol comprising the steps of:
a) Synthesizing ketene; and
b) Hydrogenating of said ketene in the presence of a catalyst to produce ethanol.

16. The method of claim 15 where the ketene is synthesized from acetic acid.

17. The method of claim 16 where the acetic acid is synthesized from methanol.

18. The method of claim 15 where the catalyst is a transition metal based catalyst.

19. The method of claim 18 where said catalyst employed in hydrogenation comprises of two or more transition metals.

20. The method of claim 19 where said catalyst employed in hydrogenation comprises a copper zinc oxide based catalyst.

21. The method of claim 19 where said catalyst employed in hydrogenation comprises a copper chromite based catalyst.

22. The method of claim 19 where the catalyst comprises of a mixture of transition metal catalysts and main group elements.

23. The method of claim 15 where the ketene is passed over a hot surface before hydrogenation to promote higher alcohol synthesis.

24. The method of claim 15 where said ketene is subjected to compression before hydrogenation to promote formation of higher alcohol products.

25. The method of claim 15 where said ketene is passed through a container with volume to increase its residence time and then hydrogenated to form higher alcohols.

26. A method to synthesize a fuel comprising the steps of:
a) Synthesizing ketene; and
b) Hydrogenating of said ketene in the presence of a catalyst to produce said fuel.

27. The method of claim 26 where said synthesized fuel is blended with a hydrocarbon mixture derived from a petroleum source.

28. The method of claim 26 where said synthesized fuel is blended with a biofuel.

29. The method of claim 26 where said synthesized fuel is blended with ethanol derived from a fermentation process.
